(19) United States
(12) Patent Application Publication
(10) Pub. No.: US 2013/0197569 A1
(43) Pub. Date: Aug. 1, 2013

(54) Nasal breathing strip having non-adherent center portion

(71) Applicant: Michael Scott Allen, Lutz, FL (US)

(72) Inventor: Michael Scott Allen, Lutz, FL (US)

(21) Appl. No.: 13/385,058

(22) Filed: Jan. 31, 2012

(51) Int. Cl. A61F 5/08

(52) U.S. Cl. 606/204.45

(57) ABSTRACT

A nasal breathing strip comprising a resiliently-deformable planar plastic strip having an inner surface adjacent a user's nose skin. Adherent portions adhere to the wearer's skin on opposite sides of the nose bridge to maintain the strip in a biased folded condition. In a first embodiment, a non-adherent central portion of the inner surface between the adherent portions is also coated with adhesive overlain by a non-adhesive material formed from paper, cloth, plastic, or combinations thereof. The non-adherent central portion pads the fulcrum function of the nose bridge and prevents irritation of skin on the bridge. Preferably the non-adhesive material is also non-allergenic for skin contact. In a second embodiment, the central portion remains free of adhesive during manufacture of the nasal strip and thus is inherently non-adherent and exposed solely to atmospheric air. Alternatively, the non-adherent central portion may be covered by a lubricating agent.

### TECHNICAL FIELD

The present invention relates to devices for enhancing human breathing through the nose; more particularly, to increase resilient devices adherent to the skin of the nose to increase the size of nasal passages, known generically in the art as “nasal breathing strips”; and most particularly, to a nasal breathing strip having a non-adherent central portion that in use is non-allergenic and non-irritating to the bridge of the nose.

### BACKGROUND OF THE INVENTION

Nasal breathing strips are well known in the art. Such a device typically employs at least one resilient planar plastic leaf spring coated with a layer of adhesive capable of adhering to the skin of a person's nose to bias the spring into a non-planar condition when the device is deformed across the bridge of a wearer's nose. Such bias acts to increase the effective cross-sectional area of the wearer's nasal passages, thus promoting greater respiratory air flow by the wearer during normal breathing cycles, and in many instances preventing snoring. See, for example, U.S. Pat. No. 5,533,503.

A serious shortcoming experienced by numerous routine users of prior art nasal breathing strips is that, with repeated use, the skin on the bridge of the nose becomes irritated, sore, and even raw from exposure to the adhesive on the central portion of the nasal strip. To alleviate this condition, such users are forced to refrain from wearing additional nasal breathing strips for a number of days sufficient to allow the condition to heal, during which time these users are denied the respiratory benefit of nasal breathing strip usage. Of course, restarting use of nasal breathing strips after healing can also start another undesirable cycle of inflammation.

A well-known manufacturer of nasal breathing strips recognizes this problem but teaches away from the present invention by offering the following online advice: “For sensitive skin, place a small drop of lotion under the centre of the strip before applying.”

What is needed in the art is a nasal breathing strip that is non-allergenic and non-chafing to the bridge of the nose.

It is a principal object of the present invention to prevent inflammation of the skin on the bridge of a nasal breathing strip wearer's nose.

### SUMMARY OF THE INVENTION

Briefly described, a nasal breathing strip in accordance with the present invention comprises a resiliently-deformable planar plastic strip having an outer surface and an inner surface adjacent a user's nose skin. The inner surface is skin-adherent adjacent to the strip ends. When the strip is folded over the bridge of a wearer's nose, the adherent portions adhere to the wearer's skin on opposite sides of the nose bridge to maintain the strip in a biased folded condition. The spring constant of the adhered strip urges the strip toward planarity, thus raising the adhered skin and opening the underlying nasal passages.

In a first embodiment, a non-adherent central portion of the inner surface between the adherent portions is also coated with adhesive overlain by a non-adhesive material formed from the group consisting of paper, cloth, plastic, and combinations thereof. The non-adherent central portion pads the fulcrum function of the nose bridge and prevents irritation of skin on the bridge. Preferably the non-adhesive material is also non-allergenic for skin contact.

In a second embodiment, the central portion remains non-coated with adhesive during manufacture of the nasal strip and thus is inherently non-adherent. The central portion is thus exposed solely to atmospheric air. Alternatively, the non-adherent central portion may be covered with a lubricating agent.

### BRIEF DESCRIPTION OF THE DRAWINGS

The present invention will now be described, by way of example, with reference to the accompanying drawings, in which:

FIG. 1 is a schematic cross-sectional view of a non-flexed generic prior art nasal breathing strip;

FIG. 2 is a cross-sectional view of the nasal breathing strip shown in FIG. 1 in flexed use position;

FIG. 3 is an exploded schematic cross-sectional view of a currently-preferred first embodiment of a nasal breathing strip in accordance with the present invention; and

FIG. 4 is a schematic cross-sectional view of a second embodiment.

Corresponding reference characters indicate corresponding parts throughout the several views. The exemplifications set out herein illustrate embodiments of the invention, and such exemplifications are not to be construed as limiting the scope of the invention in any manner.

### DESCRIPTION OF THE PREFERRED EMBODIMENTS

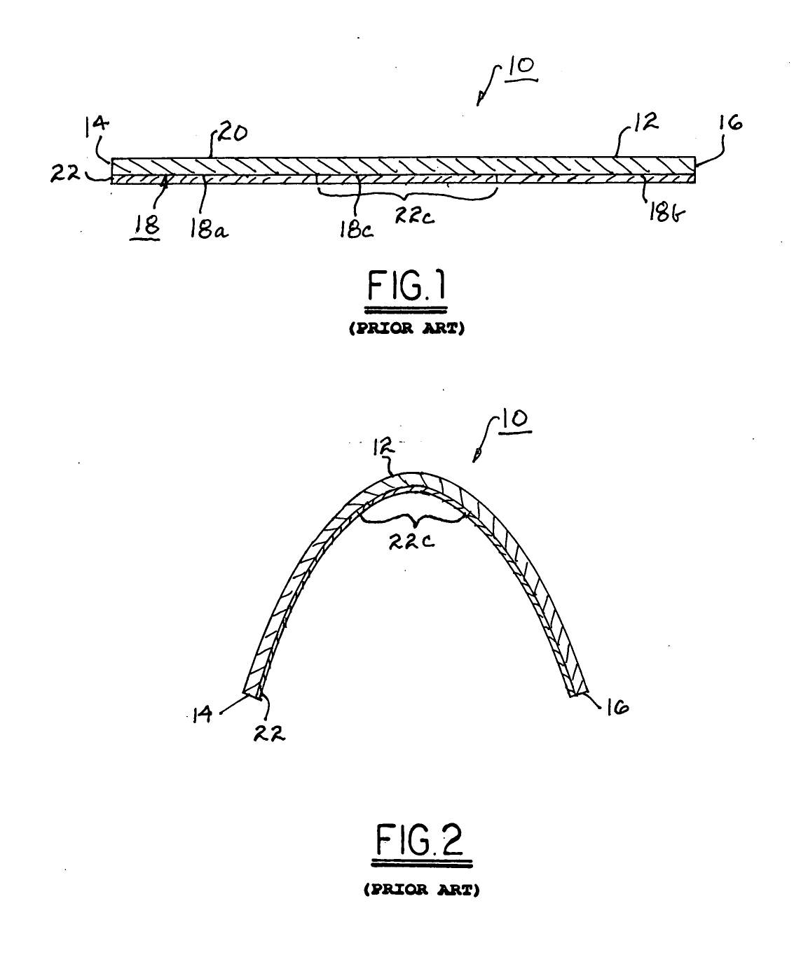

Referring to FIGS. 1 and 2, a typical generic prior art nasal breathing strip (“strip”) 10 comprises a resiliently-deformable plastic support 12 having first and second ends 14,16 and inner and outer surfaces 18,20. Support 12 is shown generically as a monolith but may include a plurality of components. In non-stressed mode, strip 10 is substantially planar. First and second surface portions 18a,18b adjacent first and second ends 14,16, respectively, and a central portion 18c are coated with a layer 22 of adhesive that is removably adherent to human skin by contact therewith. When strip 10 is folded over the bridge of a wearer's nose, first and second adherent portions 18a,18b adhere to the wearer's skin on opposite sides of the nose bridge to maintain strip 10 in a biased folded condition (FIG. 2). The spring constant of support 12 urges strip 10 toward planarity, thus raising the adhered skin and underlying tissues and opening the underlying nasal passages. Central portion 18c is adhered to the skin of the nose bridge by central adhesive portion 22c.

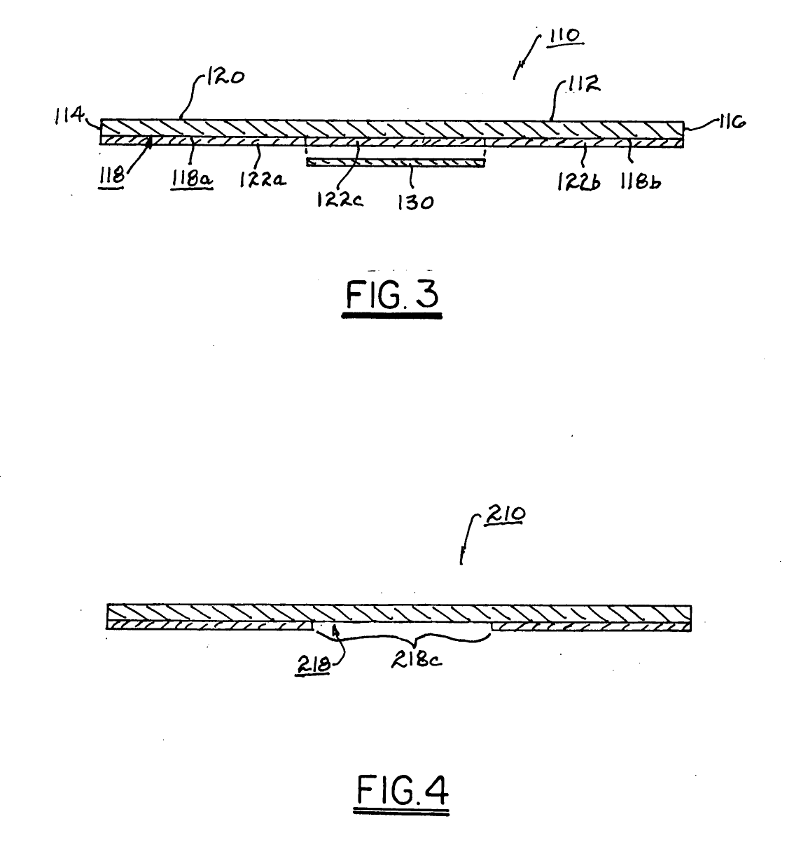

Referring now to FIG. 3, a nasal breathing strip 110 in accordance with the present invention comprises a resiliently-deformable planar plastic support 112 having first and second ends 114,116 and inner and outer surfaces 118,120, wherein inner surface 118 is defined as the surface adjacent a user's nose skin in use. Support 112 is shown generically as a monolith but may include a plurality of components. At least portions of inner surface 118, e.g., first and second inner surface portions 118a,118b adjacent to first and second ends 114,116, respectively, are coated with a layer of adhesive 122a,122b that is adherent to human skin by contact therewith, as in the prior art. When strip 110 is folded over the bridge of a wearer's nose, first and second adherent portions 122a,122b adhere to the wearer's skin on opposite sides of the nose bridge to maintain the strip in a biased folded condition as in the prior art. The spring constant of adhered strip 110 urges the strip toward planarity, thus raising the adhered skin and opening the underlying nasal passages.

In first embodiment 110 in accordance with the present invention, a non-adherent central portion 118c of inner surface 118 between first and second adhesive portions 122a,122b is also coated with adhesive portion 122c, overlain by a pad 130 of non-adhesive material formed from the group consisting of paper, cloth, plastic, and combinations thereof. Central pad 130 is non-adherent to human skin but rather serves to soften the fulcrum function of the nose bridge when strip 110 is applied and thus to prevent irritation of skin on the nose bridge. The adhesive of adhesive portion 122c may be the same as, or different from, the adhesive of adhesive portions 122a,122b.

Referring now to FIG. 4, in a second embodiment 210 in accordance with the present invention, central portion 218c of strip inner surface 218 remains non-coated with adhesive during manufacture of nasal strip 210 and thus is inherently non-adherent. In all other respects, second embodiment 210 is the same as first embodiment 110.

In a presently preferred configuration of second embodiment 210, strip inner surface 218c remains non-coated with adhesive and thus may be thought of as being covered by a boundary layer of atmospheric air. Alternatively, strip inner surface 218c may be covered by a lubricant or emollient such as body lotion (not shown).

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed is:

1. A nasal breathing strip having first and second ends, comprising:
a) at least first and second portions of a first adhesive material adjacent said first and second ends, respectively; and
b) a non-adhesive central portion formed between said first and second portions.

2. A nasal breathing strip in accordance with claim 1 further comprising a flexible plastic support having inner and outer surfaces, said inner surface defining the surface adjacent a wearer's nose during use of said strip, wherein said first and second portions are disposed on said inner surface.

3. A nasal breathing strip in accordance with claim 2 wherein said non-adhesive central portion includes a pad of non-adhesive material.

4. A nasal breathing strip in accordance with claim 3 wherein said non-adhesive material is selected from the group consisting of paper, cloth, plastic, and combinations thereof.

5. A nasal breathing strip in accordance with claim 2 further comprising a second adhesive material disposed between said pad and said inner surface.

6. A nasal breathing strip in accordance with claim 5 wherein said second adhesive material is the same as said first adhesive material.

7. A nasal breathing strip in accordance with claim 5 wherein said second adhesive material differs from said first adhesive material.

8. A nasal breathing strip in accordance with claim 2 wherein said first adhesive material is disposed discontinuously on said inner surface between said first and second ends, thereby defining said non-adhesive central portion.

9. A nasal breathing strip in accordance with claim 1 wherein said non-adherent central portion is covered with a material selected from the group consisting of lubricant, emollient, and air.
